(19) United States
(12) Patent Application Publication
(10) Pub. No.: US 2021/0353923 A1
(43) Pub. Date: Nov. 18, 2021

(54) DEVICES FOR INTRACELLULAR AND INTRATISSUE NANOINJECTION OF BIOMOLECULES AND METHODS OF PRODUCING THE SAME

(71) Applicant: Purdue Research Foundation, West Lafayette, IN (US)

(72) Inventors: Chi Hwan Lee, West Lafayette, IN (US); Dong Rip Kim, Seoul (KR)

(21) Appl. No.: 17/277,427

(22) PCT Filed: Sep. 27, 2019
(86) PCT No.: PCT/US2019/053464

§ 371 Date: Mar. 18, 2021

Related U.S. Application Data
(60) Provisional application No. 62/737,210, filed on Sep. 27, 2018.

(51) Int. Cl. A61M 37/00

(52) U.S. Cl. CPC A61M 37/0015; A61M 2205/0216; A61M 2037/0053; A61M 2037/0061; A61M 2037/0023

(57) ABSTRACT

Devices for intracellular and intratissue nanoinjection of biomolecules into a living body and methods of producing the devices. Such a device includes a flexible substrate and nanoneedles extending from a surface of the flexible substrate, and can be produced by providing a first substrate having pillars extending from a surface thereof, locally reducing diameters of the pillars at locations thereof adjacent the first substrate, embedding distal ends of the pillars in a flexible substrate, and sufficiently expanding the flexible substrate to cause the pillars to fracture at the locations thereof adjacent the first substrate and detach therefrom to define nanoneedles extending from the flexible substrate.

### CROSS-REFERENCE TO RELATED APPLICATIONS

This application claims the benefit of U.S. Provisional Application No. 62/737,210, filed Sep. 27, 2018, the contents of which are incorporated herein by reference.

### STATEMENT REGARDING FEDERALLY SPONSORED RESEARCH

This invention was made with government support under Contract No. FA2386-16-1-4105 awarded by the U.S. Air Force. The government has certain rights in the invention.

### BACKGROUND OF THE INVENTION

The present invention generally relates to intracellular and intratissue nanoinjection of biomolecules. The invention particularly relates to flexible elastomer patches having nanoneedles extending therefrom and methods of producing the same.

The ability to introduce vertically ordered silicon nanoneedles (Si NNs) into living biological systems, such as cells and tissues, enables the study of biological functions and mechanisms, thereby providing important clinical implications. Examples include nanoscale delivery of nucleic acids into cells and tissues, perturbation of cells with extracellular factors, and electrical stimulation and recording. However, current methods of delivery via Si NNs have various shortcomings due to the fabrication of the vertically ordered Si NNs on a bulk Si wafer, which is conventionally necessary as such substrate can withstand the condition of standard nanofabrication processes (i.e., high temperatures, corrosive chemicals, etc.). The intrinsically rigid, flat, and opaque Si wafer yields a large mismatch to soft, curvilinear, and see-through biological systems, thereby limiting their contact and direct observation through the Si wafer.

In view of the above, it can be appreciated that there are certain problems, shortcomings or disadvantages associated with the prior art, and that it would be desirable if an improved devices, methods, and systems were available for introducing vertically ordered NNs into living biological systems to at least partly overcoming or avoiding the problems, shortcomings or disadvantages noted above.

### BRIEF DESCRIPTION OF THE INVENTION

The present invention provides devices suitable for intracellular and intratissue nanoinjection of biomolecules into a living body and methods of producing the devices.

According to one aspect of the invention, such a device includes a flexible substrate and nanoneedles extending from a surface of the flexible substrate.

According to another aspect of the invention, a method is provided for producing a device for intracellular and intratissue nanoinjection of biomolecules into a living body. The method includes providing a first substrate having pillars extending from a surface thereof, locally reducing diameters of the pillars at locations thereof adjacent the first substrate, embedding distal ends of the pillars in a flexible substrate, and expanding the flexible substrate from a first volume to a second volume sufficient to cause the pillars to fracture at the locations thereof adjacent the first substrate and detach therefrom to define nanoneedles extending from the flexible substrate.

Technical effects of devices and methods as described above preferably include the ability to provide improved contact between nanoneedles and living biological systems, such as cells and tissue, as a result of the nanoneedles being embedded in and extending from a flexible substrate.

Other aspects and advantages of this invention will be further appreciated from the following detailed description.

### BRIEF DESCRIPTION OF THE DRAWINGS

FIGS. 1A-E include images and illustrations representing steps in methods for integrating vertically ordered nanoneedles (NNs) onto a flexible substrate. FIG. 1A shows a series of scanning electron microscopy (SEM) images (scale bar: 1 micrometer) of vertically ordered Si pillars with a passivation layer (left) and localized undercut (middle), and after the size is reduced to nanoscale (right). FIG. 1B illustrates steps for physically liberating NNs from a native Si wafer by expanding a flexible substrate formed of polydimethylsiloxane (PDMS). FIG. 1C shows an optical image (scale bar: 1.5 cm) of a representative NN-patch. FIG. 1D shows a magnified SEM image (scale bar: 20 micrometers) of the partly embedded NNs into the PDMS. The inset (scale bar: 600 nm) highlights the needle-like sharp tips. FIG. 1E shows a confocal laser scanning microscopy (CLSM) image (scale bar: 30 micrometers) of the NNs.

FIGS. 2A-C are SEM images (scale bar: 250 nm) of nanopores formed on surfaces of NNs at different treatment times.

FIGS. 3A and 3B are SEM images of NNs with varied tip size (FIG. 3A) and height (FIG. 3B).

FIGS. 4A-C contain SEM images (Scale bars: 7 micrometers, 5 micrometers, and 3 micrometers from the top) of cells interfaced with conventional Si NNs fabricated on bulk Si wafers.

FIGS. 5A and 5B contain SEM images (Scale bars: 7 micrometers and 10 micrometers from the left) of SKOV3 cells (FIG. 5A) and HDF cells (FIG. 5B) interfaced with NN-patches having different sizes of NNs.

FIG. 6 contains SEM images (Scale bars: 5 micrometers and 1.5 micrometers from the left) of NNs without nanoscale pores on their surfaces.

### DETAILED DESCRIPTION OF THE INVENTION

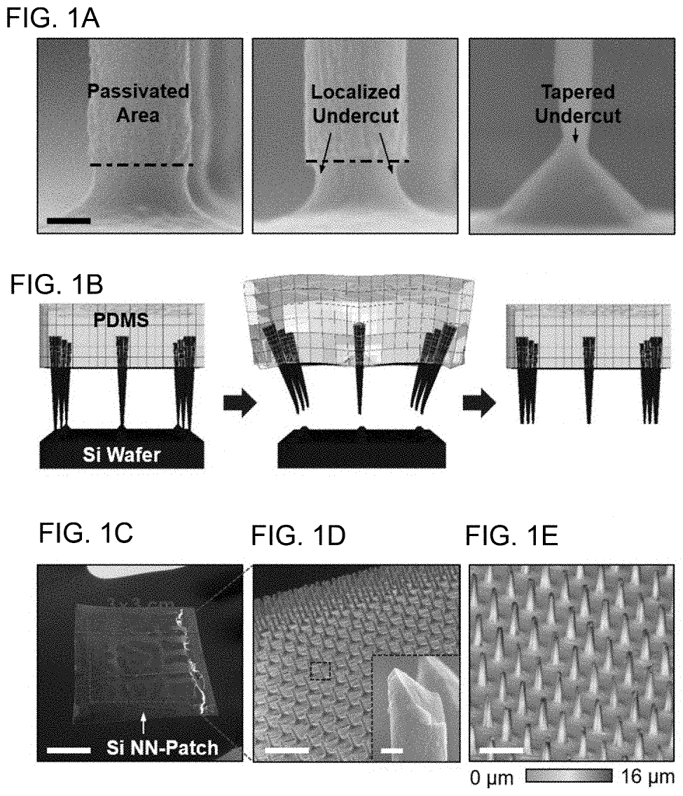

Various method steps are represented in FIGS. 1A and 1B for heterogeneously integrating vertically ordered nanoneedles (NNs) with a flexible substrate (herein referred to as an NN-patch). The NN-patch may provide an improved degree of mechanical flexibility, optical transparency, and cell and tissue compatibility relative to conventional vertically ordered NNs fabricated on a rigid wafer, such as Si NNs fabricated on bulk Si wafers. Exemplary investigations described below refer to the use of bulk Si wafers to produce Si NNs, though it is foreseeable that rigid NNs could be fabricated from other bulk materials, and NNs produced from other bulk materials and NN-patches incorporating such NNs are within the scope of this invention. Furthermore, particular treatments are described as being performed on the Si bulk wafers and NNs, though it should be understood that other processes may be suitable, particularly if the NNs are fabricated from other than bulk Si.

Referring to FIG. 1A, vertically ordered arrays of microscale Si pillars (e.g., initial diameters of about 3 micrometers) were initially fabricated on a bulk Si wafer by using standard photolithographic patterning and a deep reactive ion etching (DRIE) process (left image of FIG. 1A). Octafluorocyclobutane (C₄F₈) polymerization and DRIE processing were performed to deposit a thin passivation layer on the surface of each pillar in a selected manner (above the dashed lines in the left and middle images of FIG. 1A). A biased isotropic etching step with sulfur hexafluoride (SF₆, 85 sccm, 30 mTorr, 450 W RF Plasma power, 30 W platen power, 15 sec.) was performed to create undercuts in unpassivated surface portions of the pillars near their bases adjacent the wafer (middle image of FIG. 1A), after which a series of post-cleaning treatments with oxygen (O₂) plasma and piranha solution was performed to remove the passivation layers. The overall sizes of the pillars were then reduced via immersion of the entire structure in a solution of potassium hydroxide (KOH) at 25° C. down to nanometer scale (i.e., NNs), which simultaneously formed a tapered angle at the undercut areas (right image of FIG. 1A) such that the diameters of the pillars increased in a direction toward the distal ends (not shown) of the pillars.

Once the NNs were produced on the Si wafer, they were partly embedded into a flexible substrate and physically liberated from the bulk Si wafer by employing a transfer printing method. Suitable materials for the flexible substrate include, but are not limited to, elastomers such as polydimethylsiloxane (PDMS) or another silicone-based elastomer material such as ECOFLEX™ (commercially available from Smooth-On, Inc.) and SILBIONE® (commercially available from Bluestar Silicones), as well as other suitably flexible polymers including poly(vinyl alcohol) (PVA) hydrogels. This transfer printing method may be performed by placing a spin-casted, partly cured layer of the elastomer material at the distal portion of the as-prepared NNs such that an air gap exists between the elastomer material and the wafer (FIG. 1B, left image). A subsequent annealing step maybe used to facilitate to completion the polymerization of the elastomer material and thereby permanently embed the NNs in the flexible substrate.

As represented in the middle and right images of FIG. 1B, the NNs were physically liberated from the wafer by expanding the flexible substrate to generate cracks in the NNs that were localized at the undercut areas of the NNs where the most significant mechanical stress is concentrated. For the investigations reported herein, the entire structure was immersed in a solvent solution to cause swelling and expansion of the flexible substrate (middle image of FIG. 1B). During the swelling process, the flexible substrate underwent time-dependent non-homogeneous morphological expansion, causing a traveling wave of mechanical deformations such as bending and twisting to propagate all around and entirely through the flexible substrate. The expansion of the flexible substrate generated cracks localized at the undercut areas of the NNs where the most significant mechanical stress is concentrated. This controlled cracking phenomenon resulted in the physical separation of the NNs from the bulk Si wafer.

Dehydration of the resulting structure, for example, in a convection oven at 70° C. for about one hour, was then performed to allow the flexible substrate to recover its original volume (FIG. 1B, right image). The thickness of the flexible substrate can be then reduced by floating the structure on the surface of a wet etchant such as tetra-n-butylammonium fluoride (TBAF), followed by thorough washing with distilled (DI) water.

FIGS. 1C, 1D, and 1E show a nonlimiting example of an NN-patch produced with the method described above. FIG. 1C is an optical image of the NN-patch that depicts an array (3×3 cm) of vertically ordered Si NNs on a substrate (sheet) of PDMS. FIG. 1D is an enlarged scanning electron microscopy (SEM) image that confirms that the original geometry, gap distance, and vertical arrangement of the NNs were maintained with high-fidelity after the transfer printing process. The inset image in FIG. 1D highlights the needle-like sharp tips of the NNs that can reduce the potential damage of living biological systems during nanoinjection. FIG. 1E presents a representative confocal laser scanning microscopy (CLSM) image, indicating that the transferred NNs on the PDMS substrate have a uniform height. SEM images indicated that the fractured planes on both the donor Si wafer and the receiver PDMS substrate were uniform across the entire area.

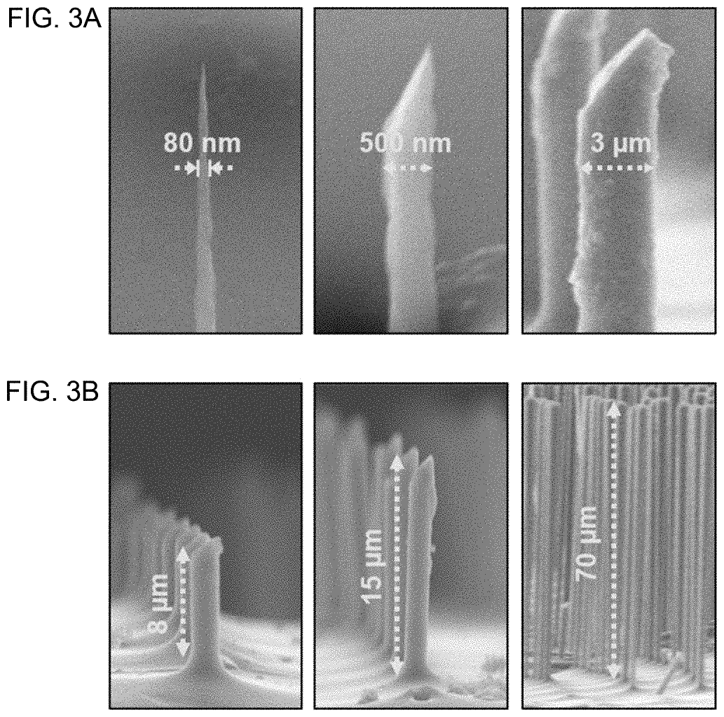

The NNs produced by the method described above included a range of tip diameters of about 80 nm to 3 micrometers, heights of about 8 micrometers to 70 micrometers, and approximate aspect ratios of about 2:1 to 125:1 (FIGS. 3A and 3B). The diameters, heights, and aspect ratios of the NNs can be varied as desired for specific intracellular and intratissue nanoinjections at various length scales. Notably, no significant change in the mechanical properties, such as Young's modulus, of the PDMS substrate was observed by the load of the NNs.

Further nonlimiting details and aspects of the investigation noted above and further nonlimiting aspects and embodiments of the invention will now be described.

The NN-patch of FIGS. 1C, 1D, and 1E was produced by initially immersing the bulk Si wafer (p-type, 525 micrometers thick, 0-100 Ω·cm) in a buffered oxide etch (BOE) solution for one minute to remove a native oxide layer on the surface. Standard photolithographic patterning and anisotropic deep reactive ion etching (DRIE) was performed to create the arrays of vertical Si pillars with a pre-defined diameter and aspect ratio. A deposition step was conducted to passivate the surfaces of the Si pillars with a thin layer of (CₓF_{y}), polymer by exploiting C₄F₈ gas with a flowrate of 130 sccm under the RF plasma power of 800 W, followed by an etching step by using SF₆ gas with a flow rate of 85 sccm under RF plasma power of 450 W and the platen power of 12 W.

Following an additional DRIE to expose the bottom areas of the Si pillars, a biased isotropic etching step was conducted to form undercuts selectively at the exposed areas by using SF₆ gas with a flow rate of 85 sccm under the RF plasma power of 450 W and the platen power of 30 W. The remaining passivation layers were removed by exploiting standard O₂ plasma treatment (20 sccm, 150 W, 50 mTorr 15 minutes) and piranha cleaning (75% H₂SO₄ and 25% H₂O₂). The entire structure was immersed in a solution of 13 wt. % potassium hydroxide (KOH) at 25° C. to reduce the overall size of the Si micropillars down to nanoscale (about 80 nm in the minimum tip size).

The as-prepared vertical NNs were then physically separated from the fabrication Si wafer and transferred to a flexible substrate in the form of a thin PDMS substrate. Control NNs were prepared separately on a Si wafer by employing similar fabrication procedures but without the transfer step. A standard isotropic etching step was used to form sharp tips on the NNs on the Si wafer under the RF plasma power of 450 W and the platen power of 0 W.

The PDMS substrate (Sylgard 184, E≈2 MPa, initial thickness about 250 micrometers) was prepared by mixing the base material and curing agent with a ratio of 10:1 by weight, followed by spin-casting at 100 rpm for ten minutes and complete polymerization at 70° C. for two hours. The PDMS substrate was then coated with another thin layer of spin-casted (about 1,000 to 2,000 rpm, ten minutes), partly cured (room temperature, three minutes) PDMS to serve as an adhesive, and the as-prepared NNs with undercuts on a Si wafer were placed upside down on the PDMS substrate.

At this stage, the thickness of the adhesive layer was controlled during the spin-casting process to determine the protruding height of NNs out of PDMS. A subsequent thermal annealing was conducted at 130° C. for ten minutes to complete the polymerization of the adhesive layer and secure the physical bonding with the surrounding NNs as well as the PDMS substrate on the bottom Immersion of the entire structure in a solvent such as hexane (20 mL) allowed the PDMS substrate to expand its volume greater than 200% within two minutes and liberate the NNs from the Si wafer via cracking at the undercut areas. The resulting structure was thoroughly washed with DI water and dehydrated in a convection oven at 70° C. for about one hour to remove the absorbed solvent, which allowed the PDMS substrate to shrink and recover its original volume. The thickness of the PDMS substrate was then reduced by floating it on a mixed etchant of 9 vol. % tetra-n-butylammonium fluoride (TBAF, 75 wt % in H₂O) and 91 vol. % acetone at the etch rate of 10 micrometers/min at room temperature. The thickness of the resulting PDMS substrate ranged from 80 micrometers to 280 micrometers.

Computational studies were performed to analyze the underlying mechanics of PDMS and NNs under mechanical deformations using the ABAQUS/standard package. The deformation of PDMS and NNs was modeled by linear elastic behavior with the Young's modulus (E) of 2 MPa and 112.4 GPa, respectively. The PDMS was modeled by a coupled displacement-temperature eight nodes solid elements (C3D8T) while the NNs were modeled by eight nodes linear brick, reduced integration solid elements (C3D8R) with pre-defined boundary conditions. Finite element analysis (FEA) was formed to evaluate the displacement for a simplified testbed structure comprising a 3×3 array of NNs built on a PDMS substrate under swelling at 100%, 170%, and 230% relative to its original volume, while assuming that the PDMS substrate swells homogeneously with time. The deformation of NNs was modeled by linear elastic behavior with the Young's modulus (E)=112.4 GPa, diameter of NNs (D)=3 micrometers, diameter of undercut area (d)=0.2 micrometers, height of NNs (H)=20 micrometers, and protruding height of NNs out of the PDMS substrate (h)=6 micrometers. The strain distribution (ε) of a single Si NN under each swelling condition revealed that the peak principal strain (εₚₑₐₖ) existed in the undercut area where the maximum magnitude, at S=230%, exceeded greater than 12% of its fracture limit (about five percent). The induced peak strain can thereby lead to the physical separation of NNs from the bulk Si wafer, as also observed in the experimental results described above. Computational (FEA) results revealed the effect of several parameters such as S, D/d ratio, and H/h ratio on the peak strain localized at the undercut area. The results indicated that the peak strain increases linearly as the PDMS substrate expands in volume (left graph) in which it rises rapidly at small D/d and then gradually reaches steady-state as the D/d ratio becomes larger. The increase of H/h ratio leads to raise the peak strain at the undercut area (right graph). Computational studies were also performed to evaluate the effect of different solvent solutions, including ethanol (S=110%), hexane (S=230%) and dichloromethane (S=280%), on peak strains.

Cell compatibility is a critical consideration for the implementation of NN-patch in many envisioned intracellular applications. For this purpose, cell compatibility assays utilized an NN-patch seeded with human dermal fibroblast (HDF) cells in a culture medium (Fibroblast Basal Medium, ATCC). This setup (i.e., NNs on bottom) allowed the cells to progressively enter into the NNs within one hour. The result obtained from a colorimetric kit (MTT, Sigma-Aldrich, USA) indicated that the proliferation rate of the cells was consistently increased throughout the assay period (72 hours), providing no significant difference when compared with that of the control NNs built on the Si wafer and a bare Si wafer without NNs. For these tests, approximately 5×10⁴ cells were seeded on a testbed NN-patch in a 24-well plate. At each measurement point, 200 μL of MTT solution was added into the wells and incubated for 4 hrs. The cell media was excavated and 400 μL of dimethyl sulfoxide (DMSO) was added to dissolve precipitated formazan. Approximately 100 mL of the solution was transferred to a 96-well plate and measured using a microplate reader at 570 nm.

To inspect for post-nanoinjection damage to cells, further tests were conducted by pressing the NN-patch onto HDF cells with a centrifuge at 500 rpm for one minute. This setup (i.e., NNs on top) allowed the NNs to be immediately injected into the cell membrane. The experimental results were obtained from a lactate dehydrogenase (LDH) assay (Abcam, USA) by exploiting the NN-patch, control NNs built on a Si wafer, and a bare Si wafer without NNs at assay periods of 24 and 48 hours. As a positive control, approximately 2.5 μL of Triton-X (Sigma-Aldrich, USA, 0.25% v:v) was introduced in the culture media to the HDF cells (blue bars). The results show that the concentration of LDH in the media remained well maintained for all of these cases, implying that no significant leakage of intracellular materials such as LDH occurred throughout the immediate nanoinjection of the NN-patch.

For the LDH assay, the cells were evaluated at 24 and 48 hours post-treatment of 0.25% v:v Triton X-100 as a positive control. After incubation, 100 mL of supernatant from each well was removed and centrifuged at 1,000 rpm for five minutes. A 10 μL of aliquot was transferred into additional microplate and 100 μL of LDH assay buffer mix was added to the wells and incubated for 30 minutes. The absorbance was measured using a microplate reader at 450 nm. For confocal microscopy analysis, the cells were fixed with 4% v:v paraformaldehyde in PBS for 15 minutes and stained with diamidino-2-phenylindole (DAPI, 500 nM) for two minutes and then mounted with an antifade reagent (Gold Antifade Mountant, Invitrogen, USA). For flow cytometry (FACS) analysis, the cells injected with GAPDH Cy3-siRNAs were trypsinized and washed with PBS several times and then fixed in 0.5% v:v paraformaldehyde for one hour. To confirm the expression of GAPDH, approximately 3×10⁴ cells were seeded on a 12-well plate and incubated for 24 hours. The efficacy in silencing GAPDH was evaluated by analyzing fluorescent intensity at 450 nm with a standard GAPDH assay kit.

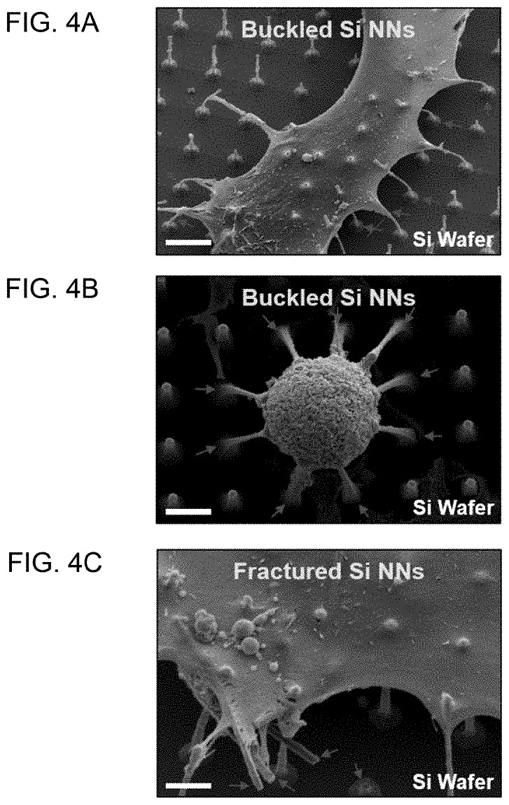

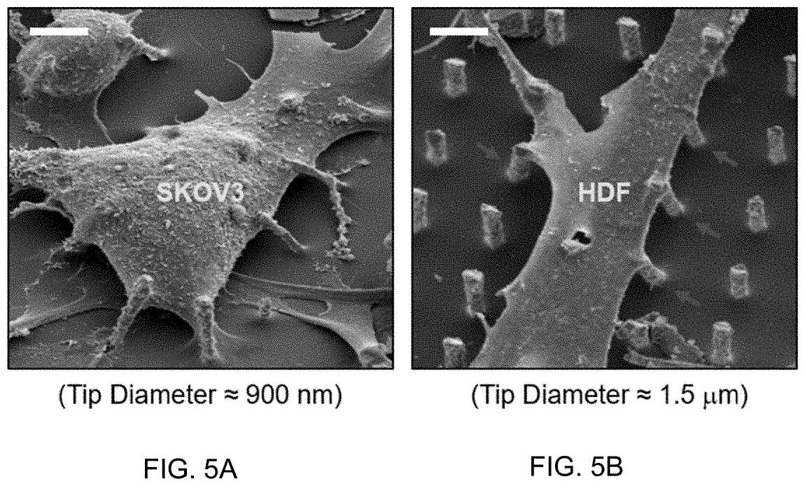

A key benefit of the NN-patch, especially when coupled with actively moving biological cells, arises from its ability to form a mechanically elastic interface between the NNs and biological cells. For instance, a soft PDMS substrate (E≈2 MPa) deforms elastically to accommodate the mechanical strains induced by the cell behaviors such as adhesion, spreading, migration, and proliferation while the stiff NNs (E≈112.4 GPa) undergo negligible deformation. As an example, using an NN-patch interfaced with MCF7 cells, the cells spread well to bridge over the gaps between adjacent NNs and induce the elongation of filopodia. Notably, the PDMS substrate deformed elastically to accommodate the strain induced by the cells, allowing the NNs to lean straightly toward cells without any mechanical buckling or fracture. These interactions were markedly distinct from those observed by exploiting conventional NNs built on a bulk Si wafer in which the NNs are either buckled out-of-plane or fractured when deformed beyond the fracture limit (FIGS. 4A-C). Control experiments on dissimilar kinds of cells such as SKOV3 and HDF by exploiting different sizes of NNs produced consistent results (FIGS. 5A and 5B), confirming that the flexible substrate was effective to prevent from the mechanical buckling or fracture of the NNs by natural behaviors of the cells.

Another beneficial feature of NN-patches that can be produced as described above includes the capability of optical transparency (about 90%), enabling the direct, real-time observation of unstrained biological cells during/after nanoinjection. Differential interference contrast (DIC) microscopy images of MCF7 cells interfaced with the NNs confirmed the following details: (1) the induced forces by the cells tend to bend the NNs toward the cells, (2) the cellular protrusions such as lamellipodia become focused near boundaries between the cells and the NNs, and (3) the cells remain viable for the entire period of intracellular nanoinjection of the NNs, exhibiting continued cell adhesion, spread, migration, and proliferation. Further investigations to understand how various types of cells would respond to the NNs that can be incorporated with drugs, genes, or proteins of particular interest would be possible with NN-patches. To show this possibility, several nonlimiting experimental demonstrations are discussed below.

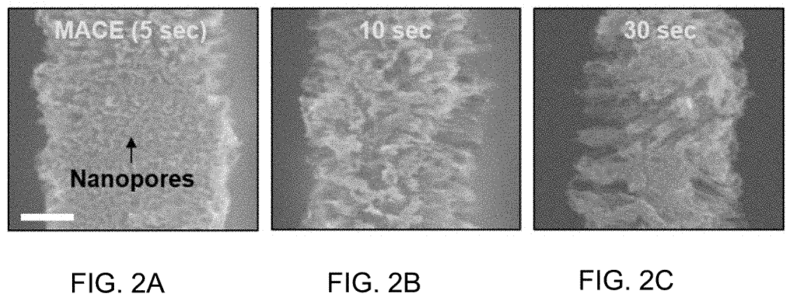

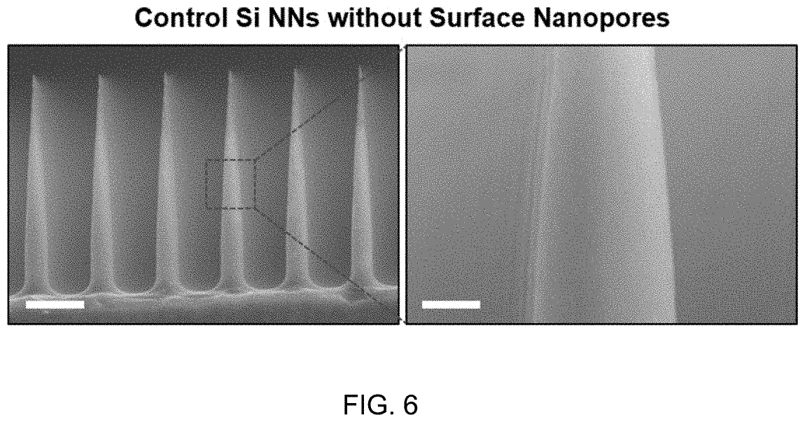

SEM images of as-fabricated NNs are shown the FIG. 6, which evidence that the NNs lack nanoscale pores (nanopores) on their surfaces. Formation of nanopores on the surface of NNs serve to significantly increase the surface areas of the NNS, which may improve the loading capacity of biomolecules. Further investigations explored a nanopore formation process that relied on a metal-assisted chemical etch (MACE) method and occurred by immersing as-prepared NNs on a bulk Si wafer in a mixed solution of catalytic silver (Ag) nanoparticles and etchants for a prescribed time period until the desired pore volume was achieved. The porosity was tuned by adjusting the molarity of solutions, doping concentration of NNs, temperature and etching time. The finished structure was then immersed in a solution of Ag etchant (TFS, KI-I2 complex liquid, Transene Inc., USA) for about one minute to eliminate the Ag residues on surfaces of NNs. The measured diameter and porosity (surface fraction) of the nanopores were ranged from about 8.5 nm to 19.4 nm and about 32.9 to 52.1 percent when the processing time for MACE was varied from 5 to 30 seconds, as shown in FIGS. 2A, 2B, and 2C.

Representative fluorescence microscopy images of the NNs with and without nanopores on surfaces thereof were obtained by exploiting a green fluorescein isothiocyanate (FITC) dye (Sigma-Aldrich, USA). The results showed that the most significant fluorescence intensity appeared at the nanoporous surfaces of the NNs, whereas the exact opposite occurred in NNs lacking nanopores. Further tests using model nucleic acids such as siRNA labeled with Cy3 produced consistent results. The nanoscale pores on surfaces of the NNs contributed to a large loading capacity of biomolecules.

To evaluate the efficacy in intracellular nanoinjection and siRNA delivery, NN-patches were tested on a range of biological cells including breast cancer cells (MCF7), human dermal fibroblasts (HDF), and ovarian carcinoma cells (SKOV3). For these tests, a testbed NN-patch was sterilized in 70% (v/v) ethanol for 30 minutes and washed twice in phosphate buffer saline (PBS) and dried under ultraviolet (UV) irradiation for one hour. The NN-patch was then immersed in a solution of two percent 3-aminopropyltriethoxysilane (APTES) for two hours to functionalize the surfaces of NNs and capture nucleic acids (Cy3-siRNA). The sequences for the siRNA used in these tests were as follows: (5′-3′)=Sense:GGAGCAGUUUGAAUGUCCAtt, Antisense:UGGACAUUCAAACUGCUCCga. Following rinsing with PBS, the cells were interfaced with the NN-patch either by seeding them over NNs (i.e., NNs on bottom) or by pressing the NN-patch over cells with centrifugal force at 500 rpm for one minute (i.e., NNs on top).

To demonstrate its utility in intracellular delivery of biomolecules, an experiment was performed by introducing the nanoporous NNs loaded with Cy3-siRNAs into model living cells such as green fluorescent protein (GFP)-expressed MCF7 cells. Confocal microscopy images were obtained for examples of the cells cultured by both seeding the cells on the NNs (i.e., NNs on bottom) and pressing the NNs on top at 500 rpm for 1 min (i.e., NNs on top). The results showed that multiple numbers of NNs were introduced into a single cell in a spatially distributed manner without any significant physical damage or cell rupture. It was also observed that the siRNAs are evenly diffused within the cytosol of the cells after 24 hours post-nanoinjection, as evidenced by confocal microscopy images. Results of a fluorescence-activated cell sorting (FACS) analysis with SKOV3 cells indicated that the transfection efficacy of siRNAs was greater than 95 percent within 24 hours post-nanoinjection. The corresponding efficacy in silencing housekeeping genes such as glyceraldehyde-3-phosphate-dehydrogenase (GAPDH) of the cells was greater than 80 percent (p<0.0001). It was also noted that no siRNA-silencing effect occurred in both the control nanoinjection of scrambled siRNAs and the treatment of the cells in the siRNA solution.

NN-patches, due to their thin and flexible properties, provide the ability to intimately contact onto the curvilinear, actively moving surface of living biological tissues such as skins and muscles. This feature is particularly important in the implementation of NN-patch in intradermal and intramuscular delivery of biomolecules. To demonstrate this, a set of in vivo evaluations was performed by exploiting a tailored size (1×1 cm) of NN-patch for an athymic nude mice model (n=10, 6 weeks old, NCr-Fox1nu, Charles Rivers Laboratories, USA). The NN-patch was comprised of nanoscale pores on the surfaces thereof that were incorporated with a fluorescent dye as a surrogate of small molecule drugs. NN-patches were placed on the skin and subcutaneous muscle of the mice for intradermal and intramuscular nanoinjections, respectively. For comparison, a control experiment was performed using conventional NNs built on a bulk Si wafer, which clearly exhibited the large morphological mismatch between the curved parts (e.g., spinal sections) of the body and the bulk Si wafer.

The results showed that the flexible NN-patch was capable of intimately interfacing with the skin in a manner that can minimize mechanical constraints on natural body motions of the mice awake. The interfacial contact between the NN-patch and the skin was durable for a long period of time (days) without any evidence of delamination, unless the mice were to tear the NN-patch off by themselves. In addition, the mice implanted with the flexible NN-patch on the subcutaneous muscle showed normal behaviors. IVIS images showed that small molecule dyes were uniformly distributed and diffused across the curved spinal regions of the mice followed by complete absorption throughout the body. These results demonstrated the potential utility of NN-patches for efficient intratissue nanoinjection of biomolecules in living bodies, including humans and animals.

For the IVIS analysis, an NN-patch loaded with DyLight 800 dye was placed on either the top of the skin or under the skin of mice, and then gently pressed down using a thumb with minimal movement of the NN-patch for at least one minute. The NN-patch was removed and washed twice with PBS. Mice were anesthetized with inhaled isoflurane anesthesia by using the Classic T3™ isoflurane vaporizer (Smith Medical, Dublin, Ohio) and exposed to 2.5% isoflurane delivered in O₂ (2 L/min) within a 1 L of induction chamber. The fluorescence of injection site was measured by using an IVIS Lumina II (Caliper Life Sciences, USA) at 30 minutes, 24 hours and 48 hours with an exposure time of one second each using a 150 W of quartz halogen lamp, filtered at indocyanine green (ICG) excitation filter (wavelength: 710-760 nm) and an ICG emission filter (wavelength: 810-875 nm).

In vivo tissue compatibility of the NN-patch in both skin-wearable and implantable scenarios was evaluated. For transdermal nanoinjections, an NN-patch was gently placed on the skin of mice awake with a medical grade adhesive and pressed firmly for about ten seconds. The behavior of the mice was real-time monitored and recorded by a high-resolution video camera. For intramuscular nanoinjections, the mice were anesthetized with avertin (Sigma-Aldrich, USA) by i.p. nanoinjection at a dose of 250 mg/kg, followed by careful incision of the skin using surgical scissors to expose the gluteal and lumbar muscles. In these tests, the incision was made on the upper-back side of the mice, wherein an NN-patch was inserted. The incisional site was then sutured using surgical needle and thread. To induce acute inflammation, a phorbol-12-myristate-13-acetate (PMA, 100 μM, 40 μL) as a positive control was rubbed on the skin, muscle and ear. After about five hours of the implementations, 100 μL of luminol ((5-amino-2,3-dihydro-1,4-phthalazinedione, 200 mg/kg BW) was administered by i.p. nanoinjection and then the mice were imaged (exposure time=3 minutes, binning=4).

Real-time bioluminescent images were obtained of the mice interfaced with the NN-patch, conventional NNs on a bulk Si wafer, and a control treatment of phorbol 12-myristate 13-acetate (PMA, 1 mM, about 20 μL) at five hours following the implementation. The images showed that no inflammation occurs in the implemented sites of the skin, subcutaneous muscle, and ear upon a systemic administration of luminol, whereas acute inflammation appears widespread in the PMA-treated mice.

For histological examination, approximately 4 micrometers of tissue section was cut and fixed in 10% formalin for 24 hours, and then stained with hematoxylin and eosin for optical inspection in a light microscope. The hematoxylin and eosin (H&E) histological cross-sectional views of the treated tissue sections produced substantial similarity with intact tissues and no signs of epidermal, dermal or capillary vessel disruption. Taken together, these findings suggested that the NN-patch can be considered as compatible for integration onto the skin and subcutaneous muscle. It is noted that Si-based nanomaterials are dissolvable in physiological circumstances, following a complete harmless resorption in the body. In these tests, the measured dissolution rate of representative nanoporous NNs was about 6 nm/day and about 68 nm/day in a phosphate buffered solution (PBS) at 37° C. with the pH of 7.4 and 10.0, respectively.

In view of the above, high-fidelity transfer printing method described herein that exploit a controlled cracking phenomenon enable the heterogeneous integration of vertically ordered NNs onto a thin flexible substrate to form an NN-patch. The NNs may be made of monocrystalline grade of Si at the nanoscale by exploiting existing Si nanofabrication technology, and thereby can create tip diameters of about 80 nm to 3 micrometers and surface pore sizes of about 5 nm to 19 nm. The resulting outcome, due to its mechanical flexibility, cell and tissue compatibility, and nanoscale controllability provides the ability to form an effective interface between the NNs and various biological systems, enabling the high-efficacy nanoinjection of biomolecules. In addition, the optical transparency of the NN-patch enables simultaneous real-time observation of unstrained cells during their interactions with the NNs. It is anticipated that this platform will impact a broad range of efforts in intracellular and/or intratissue communications, with potential applications in the areas of cell and systems biology, drug discovery, and cellular detection and manipulation.

While the invention has been described in terms of particular embodiments and investigations, it should be apparent that alternatives could be adopted by one skilled in the art. For example, an NN-patch and NNs and substrate could differ in appearance and construction from the embodiments described herein and shown in the drawings, process parameters such as treatments, temperatures and durations could be modified, expansion of the flexible substrate could be achieved by means other than swelling (e.g., thermally), and appropriate materials could be substituted for those noted. Accordingly, it should be understood that the invention is not necessarily limited to any embodiment described herein or illustrated in the drawings. It should also be understood that the phraseology and terminology employed above are for the purpose of describing the disclosed embodiments and investigations, and do not necessarily serve as limitations to the scope of the invention. Therefore, the scope of the invention is to be limited only by the following claims.

1. A device for intracellular and intratissue nanoinjection of biomolecules into a living body, the device comprising:
a flexible substrate formed of a flexible material; and
nanoneedles extending from a surface of the flexible substrate.

2. The device of claim 1, wherein the flexible material is an elastomer material.

3. The device of claim 1, wherein the nanoneedles are formed of monocrystalline silicon.

4. The device of claim 1, wherein the flexible substrate is optically transparent.

5. The device of claim 1, wherein the flexible substrate has a thickness of about 80 micrometers to about 280 micrometers.

6. The device of claim 1, wherein the nanoneedles have heights of about 8 micrometers to about 70 micrometers.

7. The device of claim 1, wherein the nanoneedles have tip diameters of about 80 nm to about 3 micrometers.

8. The device of claim 1, wherein the nanoneedles have aspect ratios of about 2:1 to 125:1.

9. A method of using the device of claim 1, the method comprising:
applying a composition to the surface of the flexible substrate from which the nanoneedles extend; and then
contacting a surface of a living body with the device such that tips of the nanoneedles extending away from the flexible substrate pierce the surface of the living body and the composition enters the living body.

10. A method of producing a device for intracellular and intratissue nanoinjection of biomolecules into a living body, the method comprising:
providing a first substrate having pillars extending from a surface thereof;
locally reducing diameters of the pillars at locations thereof adjacent the first substrate;
embedding distal ends of the pillars in a flexible substrate; and
expanding the flexible substrate from a first volume to a second volume sufficient to cause the pillars to fracture at the locations thereof adjacent the first substrate and detach therefrom to define nanoneedles extending from the flexible substrate.

11. A method of producing a device for intracellular and intratissue nanoinjection of biomolecules into a living body, the method comprising:
providing a first substrate having silicon pillars extending from a surface thereof;
locally reducing diameters of the pillars at locations thereof adjacent the first substrate;
contacting distal ends of the pillars with a partially cured elastomer material such that the distal ends of the pillars are embedded therein;
curing the elastomer material to form an elastomer substrate;
contacting the elastomer substrate with a solvent such that the elastomer substrate expands from a first volume to a second volume sufficient to cause the pillars to fracture at locations adjacent to the first substrate and detach therefrom to define nanoneedles extending from the elastomer substrate; and
returning the elastomer substrate to the first volume.

12. The method of claim 11, wherein locally reducing the diameters of the pillars comprises:
forming passivation layers on first surface portions of the pillars;
etching second surface portions of the pillars that are adjacent the surface of the first substrate and are not covered with the passivation layers;
removing the passivation layers from the first surface portions of the pillars; and then
reducing the sizes of the pillars to a nanometer scale and form tapered diameters on the pillars such that the diameters of the pillars increase in a direction from the first substrate toward distal ends of the pillars.

13. The method of claim 11, wherein the elastomer material is a silicone-based elastomer material.

14. The method of claim 11, further comprising reducing a thickness of the elastomer substrate with an etchant.

15. The method of claim 11, wherein the nanoneedles are formed of monocrystalline silicon.

16. The method of claim 11, wherein the first substrate has an optical transparency of about 90% percent or more.

17. The method of claim 11, wherein the first substrate has a thickness of about 80 micrometers to about 280 micrometers.

18. The method of claim 11, wherein the nanoneedles have heights of about 8 micrometers to about 70 micrometers.

19. The method of claim 11, wherein the nanoneedles have tip diameters of about 80 nm to about 3 micrometers.

20. The method of claim 11, wherein the nanoneedles have aspect ratios of about 2:1 to 125:1.
